(19) United States
(12) Patent Application Publication
(10) Pub. No.: US 2011/0245340 A1
(43) Pub. Date: Oct. 6, 2011

(54) Screening Method for Identifying Patients at Risk of Adverse Hepatologic Events

(76) Inventors: Russell Medford, Alpharetta, GA (US); Kenneth M. Borow, Bryn Mawr, PA (US); Hilda Maibach, Potomac, MD (US)

(21) Appl. No.: 13/036,510

(22) Filed: Feb. 28, 2011

Related U.S. Application Data
(63) Continuation of application No. PCT/US2009/055430, filed on Aug. 28, 2009.

(51) Int. Cl. A61K 31/235; G01N 33/566; C12Q 1/48; A61P 3/10; A61P 9/00

(52) U.S. Cl. 514/548; 514/546; 436/501; 435/7.92; 435/15

(57) ABSTRACT

This present invention provides methods and kits for identifying patients at risk of suffering from a drug induced liver injury, particularly for an antioxidant drug, or for identifying patients who are suffering from early stages of a liver disorder by assessing the levels of apolipoprotein in a sample of the patient and comparing that to a reference value. The reference value is predetermined by identifying a population sample and determining an upper limit of normal value. This value is then used as a reference point for comparison of apolipoprotein levels from patient samples. In one embodiment, apolipoprotein levels are combined with ATL and/or total bilirubin levels for predicting liver damage, hepatotoxicity or hepatic events after drug administration.

### CROSS REFERENCE TO RELATED APPLICATIONS

This application claims the benefit of U.S. Provisional Application No. 61/092,686, filed Aug. 28, 2008, which is incorporated herein by reference in its entirety.

### FIELD OF THE INVENTION

This present invention provides screening methods and kits for identifying patients who are at risk for liver injuries, particularly at an increased risk of showing liver toxicity upon administration of medications. The methods and kits are useful for identifying patients at risk of drug-induced liver injury to exclude such patients from certain treatment protocols.

### BACKGROUND OF THE INVENTION

Drugs sometimes cause serious injuries to the livers of patients, with loss of hepatic function leading to illness, disability, hospitalization, and even life threatening liver failure and death or need for liver transplantation. As the world population ages, more and more drugs are being prescribed and often combined with self-prescribed over-the-counter medications, so-called “dietary supplements,” special diets and alcohol. Exposure to environmental chemicals is also rising. The liver is the principal organ for metabolizing, inactivating, and disposing of all of these toxins. Their metabolites may injure the liver cells, and complex drug-drug interactions complicate the situation. The combination of all of these risk factors has increased the incidence of liver injury.

Liver injury due to prescription and nonprescription medications is a growing medical, scientific, and public health problem in the United States. In the United States, drug-induced liver injury (DILI) is now the leading cause of acute liver failure (ALF), exceeding all other causes combined (see W M Lee, et al. Acute Liver Failure Study Group). DILI is the single most common reason for Food and Drug Administration regulatory actions concerning drugs, including failure to gain approval for marketing, removal from the marketplace, and restriction of prescribing indications. Worldwide, the estimated global annual incidence rate of DILI is 13.9-24.0 per 100,000 inhabitants, and DILI accounts for an estimated 3%-9% of all adverse drug reactions reported to health authorities (see Aithal G P, et al. (1999) Br Med J 319: 1541-5; Friis and Andreasen (1992) J Intern Med 232: 133-138; and Dossing and Anderson (1982) Scan J Gastroenterol 17:205-211).

Hepatotoxicity has been consistently the most important single cause of withdrawals and marked limitations of use of drugs or refusal to approve them. In the 1950's, iproniazid (Marsilid) was probably the most hepatotoxic drug ever marketed, but isoniazid, from the same period, has been found to cause serious hepatotoxicity in about 0.1% of recipients. Benoxaprofen (Oraflex), ticrynafen (Selacryn), bromfenac (Duract) and troglitizone (Rezulin) all were withdrawn because of hepatotoxicity, and ibufenac, perhexilene and dilevalol, all marketed abroad, were never approved in the United States because of this issue. Hepatotoxicity has also caused important limitations of use for many drugs, including isoniazid, labetalol, dantrolene, felbamate, pemoline, tolcapone, and trovafloxacin.

While in most cases, hepatotoxicity is recognized late, as the incidence is often low or dependent on other circumstances and neither animal nor human experience before marketing yields recognized signals of hepatotoxic potential. Post-marketing surveillance now detects serious hepatotoxins in months (bromfenac, tolcapone, troglitizone, trovafloxacin), in marked contrast to the years of delay in the past (iproniazid, isoniazid), but it is obviously preferable to discover them before marketing.

The use of complementary and alternative medicines has also been increasing in Western countries, and there are numerous reports of hepatotoxicity from such products in both animal models as well as in humans (see Zimmerman H J. (1999) Hepatotoxicity: the Adverse Effects of Drugs and other Chemicals on the Liver, 2nd ed. Lippincott Williams & Wilkins, Philadelphia, Pa., 1999, pp. 731). Examples of alternative products that have a well-established potential to cause liver injury include pyrrolizidine alkaloids (Comfrey), chaparral leaf, germander, pennyroyal (squawmint oil), mistletoe, kava, and weight-loss preparations containing usnic acid (Favreau J T, et al. (2002) Ann Intern Med 136:590-5).

It has become clear that certain individuals are much more susceptible to drug-induced liver damage than are others, and uncommon but severe idiosyncratic liver damage requires special consideration as a safety problem. Not only are people genetically diverse, which affects the way they metabolize drugs and other chemicals, but each person's life experience is different. Drug-induced liver injury is the leading cause of acute liver failure among patients presenting for evaluation at liver transplant centers in the United States, and the leading single cause for having to remove approved drugs from the market.

The ability of certain signals for identifying patients who are suffering from DILI has been suggested, notably transaminase elevations of various degrees (3 fold, 5 fold, etc.) and frequencies (2%, 3%, etc.) and serum transaminase elevations accompanied by elevated bilirubin (see Zimmerman (1978) Drugs 16:25-45). Generally, patients provide, at regular intervals, serum samples for testing activities of alanine aminotransferases (ALT, also known as SGPT), aspartate aminotransferases (AST, also known as SGOT), alkaline phosphatase (ALP), total bilirubin (Bt), serum albumin and, much less commonly, blood prothrombin time. However, although these markers may be useful for identifying drugs that should not be approved or should be closely monitored, these signals are not useful for predicting the patients who should be excluded from receiving a potentially hepatotoxic drug before putting those patients at risk for an adverse event.

Preclinical studies often fail to predict liver toxicity levels, particularly those of low incidence or of lesser severity Animal studies, although useful in identifying severe toxicities, fail in predicting these rare hepatic events. The situations that cause individuals to be at increased risk for a DILI are likely due to combinations of environmental factors that are impossible to recreate in a laboratory, such as drug compliance, additional or alternative medicine combinations, environmental exposures and genetic predisposition. It has been shown repeatedly that animal models simply fail to account for these variables.

Although the pre-approval clinical phases of drug development represent a key arena for identification of hepatotoxic potential, clinical trials may not present any evidence of liver toxicity if the testing population is too small, too limited, or generally not representative of the subject populations that are ultimately exposed to the drug. At this stage, drugs with significant toxic potential are easily identified but those with low toxic potential may be less easily recognized. As a general rule, if an overt adverse event occurs in 1 per 1000 people, a study must include at least 3000 individuals, which is a typical pre-approval size. An incidence of less than 1 per 1000 may never appear in the pre-approval setting. Furthermore, if the adverse reaction is delayed, the clinical trials may have included a far smaller number of exposed individuals at risk for sufficient duration. Furthermore, the frequency of testing and rules for stopping the drug may confound the identification of a significant signal.

It is recognized that pre-existing liver disease is likely a risk factor for DILI. However, reliable indicators of liver disease that could predict subjects at risk are lacking. Subjects with severe liver disease usually are excluded from clinical studies, but subjects with mild elevations of biomarkers such as serum alanine aminotransferase (ALT) (in the range of 2-3 times the upper limit of normal, i.e. subjects with mild liver disease) are usually included. ALT values are not predictive of DILI because in general such patients are not at increased risk of suffering idiosyncratic drug reactions, although their hepatic response to an idiosyncratic reaction may be exaggerated. The FDA has emphasized that aminotransferase abnormalities that are less than 3× ULN are common in untreated and placebo-treated subjects and are not informative about the potential for the development of severe DILI. Therefore, it has become standard practice to look at greater deviations such as aminotransferase values greater than 3×, greater than 5×, or greater than 10× upper limit of normal (ULN). Because these abnormalities can occur in placebo-treated groups, it is important to compare their rate in drug-exposed subject groups relative to control groups looking for an increased rate of aminotransferase elevation throughout the overall study population compared to control” (see FDA Guidance Document on DILI).

Most significant hepatotoxins cause predominantly hepatocellular injury indicated by leakage of ALT from injured liver cells without prominent evidence of hepatobiliary obstruction. The ability to cause some hepatocellular injury is not a reliable predictor of a drug's potential for severe DILI. Many drugs that cause transient rises in serum aminotransferase activity do not cause progressive or severe DILI even if drug administration is continued. Many drugs show increased ALT signal without conferring a risk of severe injury (e.g., tacrine, statins, aspirin, heparin) indicating low specificity for an excess of aminotransferase elevations alone. It is only those drugs that cause hepatocellular injury extensive enough to affect the liver's functional ability to clear bilirubin from the plasma or to synthesize prothrombin and other coagulation factors that cause severe DILI.

There remains a need for a test that can readily predict patients who are at risk of suffering from DILI, particularly DILI of low severity or frequency, before administration of a potentially toxic drug.

An object of the present invention is to provide methods and kits for identifying patients at risk of DILI and for providing treatment regimens that incorporate such information.

### SUMMARY OF THE INVENTION

The present invention is founded on the recognition that levels of certain lipoproteins in a patient can be used to predict the risk that a patient will develop a drug induced liver injury after administration of a drug. In particular, levels of apolipoprotein A1 (ApoA1) in serum relate to this risk. Redox signaling pathways play an important role in both normal and pathological cell function in tissues including the liver. Numerous studies suggest that the regulation of these signals may underlie the molecular and biochemical pathogenesis leading to clinical liver disease. Although the liver has a significant reserve capacity, over time, this reserve capacity can be diminished by various stress conditions including underlying disease states such as type 2 diabetes mellitus or atherosclerosis and exposure to both pharmaceuticals and environmental toxins. Each of these factors, taken in isolation, may have no obvious adverse effect on the liver. However, a combination of stress factors occurring simultaneously may overcome hepatic reserve capacity resulting in hepatocyte injury.

The present specification provides evidence that patients at risk for liver injury exhibit a compensated hepatic response, particularly a compensated response to oxidant signals, even in the absence of clinically defined laboratory abnormalities typically associated with liver injury. With otherwise normal laboratory values, modest elevations in ApoA1 expression to concentrations greater than the upper limit of normal can reflect a compensated hepatic stress response that identifies individual patients at risk for progression to hepatic abnormalities in response to pharmacological agents.

Therefore, in one embodiment, a method of identifying a patient at risk of a liver injury, and in particular a drug-induced liver injury, is provided comprising 1) measuring the level of ApoA1 in a bodily fluid from the patient; and 2) comparing the measured level of ApoA1 in the sample with a reference measurement in a population. In certain instances, a patient who has one or more samples in which the measured level of ApoA1 is greater than an upper limit of normal (ULN) in a reference population is considered at increased risk of liver injury, in particular at greater risk of drug-induced liver injury. In some embodiments, the drug induced liver injury is from an antioxidant drug. In certain other instances, the drug induced liver injury is from a drug that increases PPAR activity. In particular embodiments, if the measured level of ApoA1 in the bodily fluid is less than or equal to the ULN, a pharmacological agent is administered to the patient and if the measured level of ApoA1 is greater than the ULN, a pharmacological agent is not administered.

In certain embodiments, measurement of ApoA1 or structural modifications of ApoA1 are associated with redox related conditions, and in particular inflammatory conditions. In certain instances, the measurement is related to disorders such as diabetes. In some embodiments, the measurement is of levels of ApoA1. In other embodiments, the measurement is of ApoA1 structural modifications such as lipid modification. In some embodiments, the ApoA1 measurement above a ULN indicates a patient is in need of treatment for a condition.

In some embodiments, the methods further comprise measuring a level of ALT in a sample from the patient and comparing that to a reference ALT level in the population. In these instances, a measurement of ALT that exceeds ULN is also used to classify the patients as at increased risk of an adverse liver injury. In certain instances, the measurement of ALT is taken before any drug is administered. In these instances, the ALT level can be used as a further exclusionary criteria to identify patients who are at increased risk of a drug induced liver injury. In some instances, an ALT level of greater than ULN is identified, but in other instances an ALT level of at least 1.5 or at least 2.0 or greater ULN is provided as criteria for exclusion. In certain instances, ALT levels are measured in a patient receiving a drug after a period of time, such as at one week, two weeks, three weeks, four weeks, five weeks or more after commencing a therapeutic regimen. Patients whose ALT level is measured as exceeding ULN may be considered at increased risk of developing, having, or suffering from, liver toxicity. In particular embodiments, if the measured ALT level is less than or equal to the ULN, a pharmacological agent is administered to the patient and if the measured ALT level is greater than the ULN, a pharmacological agent is not administered.

A patient who has one or more samples in which measured ApoA1 level exceeds the reference, such as a ULN in a population, may also be considered to have increased inflammatory activity. Such a patient may be considered at risk for additional disorders, including inflammatory disorders such as rheumatoid arthritis. In certain instances, the patient is at risk of or suffering from a disorder in glucose metabolism. Such a disorder may be diabetes mellitus and in particular may be type 2 diabetes mellitus.

In certain instances, a treatment protocol will be designed based on the results of an ApoA1 measurement. This treatment protocol may require that antioxidant drugs not be given to a patient whose ApoA1 measurement exceeds the reference value, such as an ULN, or it may be that the patient is closely monitored for hepatotoxicity. In addition, the treatment protocol may require an adjustment in external factors, such as diet or exercise, to decrease additional exposure to environmental toxins that may exacerbate liver injury.

In another embodiment, a method of identifying patients for drug treatment is provided comprising measuring ApoA1 levels in a bodily fluid; comparing the measured value with a reference measurement of ApoA1 levels in a population; and only providing drug treatment if ApoA1 levels in the fluid are less than or equal to the reference value. In certain instances, the reference value is an ULN in a reference population.

In specific embodiments, the patient sample is a serum sample. In other embodiments, the sample is a plasma sample.

In some embodiments, the ULN is about 165 mg/dL of ApoA1 in the sample. In other embodiments it is between 150 and 200 mg/dL, between 155 and 195 mg/dL, between 160 and 190 mg/dL, between 165 and 185 mg/dL. In some embodiments, a patient is considered at risk of liver injury if the measured level of ApoA1 is greater than 150 mg/dL or greater than 155 mg/dL or greater than 160 mg/dL or greater than 165 mg/dL or greater than 170 mg/dL.

In yet another embodiment, a kit is provided for identification of a patient at risk of a drug induced liver injury comprising a detection system to measure a level of ApoA1 in a patient sample and a system to compare the measured levels to a normal level in a population.

In some embodiments, the detection system can be a labeled antibody to ApoA1 or an ELISA kit comprising a measuring antibody to ApoA1 and a labeled secondary antibody. In other embodiments, the detection system can be a binding partner other than an antibody to ApoA1. In yet further embodiments, the detection system can detect levels of the ApoA1 gene product, such as by RT-PCR. The comparison system can be a separate detection kit in which the level of ApoA1 is standardized to correspond to an upper limit of normal. The readout can be on a colorimetric scale or can be based on a direct comparison of the level of signal from the detection systems. In other embodiments, a chart is included in the kit that allows comparison of the measured ApoA1 levels in the sample with an upper limit of normal in the population. In certain instances, a visual readout is included which provides a marker if the ApoA1 level in the sample is greater than 1.0 times the upper limit of normal. In specific embodiments, the kit includes a detection apparatus that provides a marker if the measured level of ApoA 1 in the sample is greater than 165 mg/dl.

The methods and kits of the present invention may also be useful for monitoring and diagnosing various liver diseases, including early stage tissue injury/organ rejection, certain forms of viral infection, drug toxicity, and alterations in liver function. The methods provide information not currently available in the clinical arena, and are rapid and reproducible. The methods and kits are especially useful to evaluate therapeutic agents and drugs for their toxicity with respect to liver damage. The early detection of liver disease by the methods of the present invention can additionally permit earlier clinical intervention if adverse reactions do occur.

In one aspect, the present invention provides a method for detecting liver damage or potential for liver damage in a subject by measuring an ApoA1 level in a sample from the subject and comparing the level to a normal level. If the ApoA1 level exceeds an upper limit of normal (ULN), then the patient is considered at greater likelihood of suffering from or at risk of suffering from liver damage. Such early diagnosis can be useful in providing motivation for early intervention and to provide information to analyze any proposed medical regimens.

### BRIEF DESCRIPTION OF THE FIGURES

FIG. 1 is a graph of Hazard rate for Liver injury as a function of ApoA1 levels in a patient population and shows age-adjusted effect for the 5th to 95th percentile range of baseline ApoA1 on subsequent liver events using a Cox Proportional Hazards Model. As a point of reference, the ULN for ApoA1 was 165 mg/dL for this trial. The solid line and the dashed line represent AGI-1067 and placebo data, respectively.

### DETAILED DESCRIPTION OF THE INVENTION

The present invention provides methods and compositions useful, e.g. for clinical screening, diagnosis and prognosis of liver response in a mammalian subject, for monitoring the results of liver response therapy, for identifying patients most likely to have an adverse response to a particular therapeutic treatment and for drug screening and drug development. In a particular embodiment, the invention provides methods for determining patients who are at risk for developing drug-induced hepatotoxicity. For clarity of disclosure, and not by way of limitation, the invention will be described with respect to the analysis of blood or liver tissue samples. However, as one skilled in the art will appreciate, based on the present description, the assays and techniques described herein can be applied to other types of samples containing lipoproteins, including a body fluid (e.g. blood or a fraction of blood comprising serum or plasma or both, spinal fluid, urine or saliva), a tissue sample from a subject at risk of having or developing a liver response (e.g. a biopsy such as a liver biopsy) or homogenate thereof.

As used herein the phrase “adverse hepatologic event” or the like includes hepatotoxicity, liver damage and liver disease.

In certain embodiments, the methods and kits of the invention are useful in identifying patients at risk of adverse events in response to anti-oxidant agents.

Plasma lipoproteins are carriers of lipids from the sites of synthesis and absorption to the sites of storage and/or utilization. Lipoproteins are spherical particles with triglycerides and cholesterol esters in their core and a layer of phospholipids, nonesterified cholesterol and apolipoproteins on the surface. They are categorized into five major classes based on their hydrated density as very large, triglyceride-rich particles known as chylomicrons (less than 0.95 g/ml), very low density lipoproteins (VLDL, 0.95 to 1.006 g/ml), intermediate-density lipoproteins (IDL, 1.006 to 1.019 g/ml), low-density lipoproteins (LDL, 1.019 to 1.063 g/ml) and, high-density lipoproteins (HDL, 1.063 to 1.210 g/ml). Plasma lipoproteins can be also classified on the basis of their electrophoretic mobility. HDL co-migrate with alpha-globulins, LDL with beta-globulins, VLDL between alpha and beta-globulins with so called pre-beta globulins, whereas chylomicrons remain at the point of application. (Osborne, J. D. and Brewer, B. Jr. Adv. Prot. Chem. 31:253-337 (1977); Smith, L. C. et al. Ann. Rev. Biochem., 47:751-777 (1978)).

Apolipoproteins are protein components of lipoproteins with three major functions: (1) maintaining the stability of lipoprotein particles, (2) acting as cofactors for enzymes that act on lipoproteins, and (3) removing lipoproteins from circulation by receptor-mediated mechanisms. The four groups of apolipoproteins are apolipoproteins A (Apo A), B (Apo B), C (Apo C) and E (Apo E). Each of the three groups A, B and C consists of two or more distinct proteins. These are for Apo A: Apo A-I, Apo A-II, and Apo A-IV, for Apo B: Apo B-100 and Apo B-48; and for Apo C: Apo C-I, Apo C-II and Apo C-III. Apo E includes several isoforms. These apolipoproteins are contemplated for use in the methods described herein.

Apo A-I is the major protein constituent of lipoproteins in the high density range. Apo A-I may also be the ligand that binds to a proposed hepatic receptor for HDL removal. A number of studies support the clinical sensitivity and specificity of Apo A-I as a negative risk factor for atherosclerosis (Avogaro, P. et al., Lancet, 1:901-903 (1979); Maciejko, J. J. et al., N. Engl. J. Med., 309:385-389 (1983)). Some investigators have also described Apo A-I/Apo B ratio as a useful index of atherosclerotic risk (Kwiterovich, P. O. et al., Am. J. Cardiol., 69:1015-1021 (1992); Kuyl, J. M. and Mendelsohn, D., Clin. Biochem., 25:313-316 (1992)).

ApoA1 comprises 65% of the apolipoprotein of high density lipoprotein (HDL), providing the structural scaffold for its formation. It is also a co-factor for lecithin cholesterol acyl transferase (LCAT), required for esterification of cholesterol to cholesteryl esters. HDL-cholesterol is involved in the reverse transport of cholesterol from peripheral tissues to the liver, from where it can be excreted. Hence ApoA1 deficiency confers increased risk of coronary artery and peripheral vascular disease, even in the absence of other coronary risk factors. Patients with significant arteriosclerosis generally have lower plasma ApoA 1 concentrations than a normal population. Specific genetic abnormalities of the ApoA1 gene may be associated with reduced levels of ApoA1 and HDL. Reduced ApoA1 values are also associated with smoking, diets rich in carbohydrates and/or polyunsaturated fats, dyslipoproteinaemias (eg familial hypo-alphalipoproteinaemia), uncontrolled diabetes, liver disease, chronic renal failure, and some therapies (beta blockers, diuretics, progestins, androgens).

Raised ApoA1 concentrations are associated with pregnancy, familial hyperalphalipoproteinaemia, and with drugs such as carbamazepine, phenytoin, phenobarbitone, oestrogens, oral contraceptives, ethanol, niacin, fibrates and statins. Most genetic hypoalphalipoproteinaemias are caused by mutations in enzymes, and transporters involved in reverse cholesterol transport. Mutations in ApoA1 are rare and associated with amyloidosis, peripheral neuropathy and both increased and decreased risks of atherosclerosis.

Transcriptional regulation of ApoA1 is upregulated by the peroxisome-proliferator activated receptor α (PPAR-α). Within the cell, PPAR-α is activated by ligands such as oxidized free fatty acids that not only mediate cellular redox signalling but also represent hepatocellular responses to stress. ApoA1 has well established anti-oxidant and anti-inflammatory activities both in vitro and in vivo that can serve to inhibit redox sensitive signals driving its hepatic expression. It has now been recognized that the presence of modestly elevated ApoA1 levels define a patient subgroup, especially in type 2 diabetes mellitus, characterized by endogenous antioxidant and anti-inflammatory compensation to hepatic stress associated with low level inflammatory, oxidant and/or pharmacologic challenges. Drugs that confer additional oxidant-like stress that cannot be further compensated by the liver can therefore lead to hepatocyte injury, or drugs that augment endogenous direct and indirect antioxidant mechanisms may lead to overcompensation.

ApoB-100 is an integral component of the four major atherogenic lipoproteins: VLDL, IDL, LDL and Lp(a). Apo B-100 is distinguished from Apo B-48, which is found only in lipoproteins of intestinal origin, such as chylomicrons and chylomicron remnants. Apo B-48 is usually undetectable in the systemic circulation, except in rare subjects with Type I, III, or V hyperlipidemia. Apo B's initial function in VLDL and IDL appears to be structural; however, with exposure of binding domains on LDL, it becomes responsible for interaction with high-affinity LDL receptors on cell surfaces, which results in uptake and removal of LDL from the circulation. Several studies have shown that an increased Apo B level in blood is a reliable marker for coronary atherosclerosis (Sniderman, A. et al., Proc. Natl. Acad. Sci. USA, 77:604-608 (1980); Kwiterovich, P. O. et al., Am. J. Cardiol., 71:631-639 (1993); McGill et al. Coron. Artery Dis., 4:261-270 (1993); Tornvall, P. et al., Circulation, 88:2180-2189 (1993)).

Techniques used for both Apo A-I and B include immunological procedures using antibodies directed against Apo A-I or B and include radio-immunoassay (RIA), enzyme immunoassay (ELISA), competitive or capture systems, fluorescence immunoassay, radial immunodiffusion, nephelometry, turbidimetry and electroimmunoassay.

Kits and methods using antibodies which are immunoreactive with specific apolipoproteins are used to determine the concentrations of apolipoproteins such as ApoA1 in human blood, serum or plasma sample to determine an individual's risk of an averse hepatic event after administration of a drug. Useful monoclonal antibodies (MAbs) that may be used in these kits and methods are described for example in U.S. Pat. No. 7,098,036 that specifically bind to epitopes present in apolipoproteins and lipoproteins, enabling rapid and reliable determinations of levels of specific blood lipoprotein and/or apolipoprotein levels, including Apo B-100, Apo A-I, Apo A-II, Apo C-III, and Apo E.

Serum ApoA1 (and ApoB) levels are increasingly recognized as better indicators of atherosclerotic risk than cholesterol and triglycerides alone. Atherosclerotic patients are better distinguished from normal individuals by the finding of increased plasma ApoB or decreased plasma ApoA1 than by a raised LDL- and low HDL-cholesterol. The ratio of ApoA1 to ApoB is considered to provide a particularly good index of cardiovascular risk as compared to the individual values.

### Methods

The present invention encompasses methods of determining or predicting if a drug, compound, or other therapeutic agent for use in the treatment for a disease or other medical condition will be likely to have hepatotoxic effects, e. g. idiosyncratic hepatotoxicity, in vivo. Ideally, such methods are performed prior to administration of the drug (or combination of drugs) to a patient or patient population.

In one aspect, the present invention provides a method for detecting liver damage in a subject by measuring an apolipoprotein level such as ApoA1 level in a sample from the subject and comparing the level to a normal level. If the apolipoprotein level exceeds an upper limit of normal level, then the patient is considered at greater likelihood of suffering from or at risk of suffering from liver damage. Such early diagnosis can be useful in providing motivation for early intervention, and to provide information to analyze any proposed medical regimens. Certain subjects may also be excluded from treatment with a drug if they demonstrate a risk of liver damage.

In certain embodiments, measurements of apolipoprotein such as ApoA1 are supplemented by measurements of ALT and total bilirubin, to identify patients currently suffering from hepatic events. As used herein, the term ULN refers to a predetermined apolipoprotein level that is identified from normal individuals in a population. In related embodiments, ULN may be measured from a sample of individuals in a geographic region, ethnic population or defined by other criteria. The population is then used to identify a threshold ULN for later comparison to an apolipoprotein level from a test patient. The ULN is the threshold value within which 95 percent of a healthy normal population falls and is thus determined as the value by which 5% or 5% or less of the normal population exceeds this value.

As referred to herein, the term “population” or “patient population” refers to a group of two or more patients. The patient population can be a few patients, a dozen patients, hundreds of patients, or thousands of patients. The population can be defined as patients in need of treatment for a particular condition, for example diabetes or atherosclerosis. The population can be, for example, those involved in a study wherein some patients are administered a therapeutic agent and others are administered a placebo. The term “patient population” is not meant to be limiting. For example, the term also encompasses a “reference population” or two or more people who will not undergo treatment for a condition.

In certain instances, elevations of ALT above 1.5 times ULN, above 2 times ULN, above 2.5 times ULN, above 3 times ULN, above 3.5 times ULN, above 4 times ULN, above 4.5 times ULN, or above 5 times ULN are diagnostic of hepatic events. In certain other instances, total bilirubin levels (TBL) of greater than 1 times ULN, greater than 1.5 times ULN and in particular greater than 2 times ULN are also diagnostic of hepatic events. In particular, the combination of ALT and TBL above ULN are diagnostic of hepatic events.

In certain embodiments, a patient is categorized as at risk if the measured apolipoprotein level exceeds 1.0 of the upper limit of normal (ULN) in a population, or exceeds 1.1 ULN, or 1.2 ULN, or 1.3 ULN, or 1.4 ULN, or 1.5 ULN in a population. In certain embodiments, the ULN is measured in a geographic population. In certain other embodiments, the ULN is measured in a sample of individuals having a disorder. In particular, in certain embodiments, the ULN is measured based on a measurement of individuals having diabetes. In certain embodiments, these patients are diagnosed based on a glycemic parameter, such as a glucose level above 7.0 mmol/L, or a hemoglobin A1c (HbA1c) value greater than 7%.

In other embodiments, the patient is diagnosed as at risk if the measured apolipoprotein level exceeds at least one standard deviation from normal. In certain instances, this can be at least 1 or at least 1.5 or at least 2 or greater standard deviations. The standard deviation can be calculated based on a sample of at least 100 or at least 500 or at least 1000 individuals.

Different apolipoproteins have different reference levels based on their normal serum concentration. In a particular embodiment, the reference level (also the ULN) of ApoA1 is about 150 mg/dL, about 155 mg/dL, about 160 mg/dL, about 165 mg/dL, about 170 mg/dL, about 175 mg/dL, about 180 mg/dL, about 185 mg/dL or about about 190 mg/dL. In other subembodiments, the reference level is between 150 and 200 mg/dL, between 155 and 195 mg/dL, between 160 and 190 mg/dL, between 165 and 185 mg/dL. greater than 150 mg/dL or greater than 155 mg/dL or greater than 160 mg/dL or greater than 165 mg/dL or greater than 170 mg/dL.

In a particular embodiment, the reference level of Apo-A-II is about 30 mg/dL, about 35 mg/dL, about 40 mg/dL, about 45 mg/dL, or about 50 mg/dL. In other embodiments, the reference level is between 10-50 mg/dL, between 20-40 mg/dL or greater than 50 mg/dL.

In a particular embodiment, the reference level of Apo-A-IV is about 30 mg/dL, about 35 mg/dL, about 40 mg/dL, about 45 mg/dL, or about 50 mg/dL. In other embodiments, the reference level is between 10-50 mg/dL, between 20-40 mg/dL or greater than 50 mg/dL.

In a particular embodiment, the reference level of Apo-B is about 120 mg/dL, about 125 mg/dL, about 130 mg/dL, about 135 mg/dL, or about 145 mg/dL. In other embodiments, the reference level is between 100-150 mg/dL, between 120-140 mg/dL or greater than 150 mg/dL.

In a particular embodiment, the reference level of Apo-C-II is about 5 mg/dL, about 7 mg/dL, about 8 mg/dL, about 10 mg/dL, or about 15 mg/dL. In other embodiments, the reference level is between 3-8 mg/dL, between 4-6 mg/dL or greater than 10 mg/dL.

In a particular embodiment, the reference level of Apo-C-III is about 10 mg/dL, about 12 mg/dL, about 15 mg/dL, about 17 mg/dL, or about 20 mg/dL. In other embodiments, the reference level is between 5-15 mg/dL, between 8-12 mg/dL or greater than 20 mg/dL.

In a particular embodiment, the reference level of Apo-E is about 5 mg/dL, about 7 mg/dL, about 8 mg/dL, about 10 mg/dL, or about 15 mg/dL. In other embodiments, the reference level is between 3-8 mg/dL, between 4-6 mg/dL or greater than 10 mg/dL.

### Methods of Detection

In certain embodiments, levels of apolipoprotein can be measured in serum or plasma or other bodily fluid samples from the patient. The apolipoprotein levels can be measured by any suitable means, but for example, can be measured using an antibody to an epitope of the apolipoprotein protein. In some embodiments, the levels of apolipoprotein are measured using an antibody assay such as ELISA. ELISA kits for quantitative determination of native and recombinant human apolipoprotein in plasma or serum samples are commercially available, such as from Mabtech AB. Such a kit can contain a capture Ab, such as a monoclonal antibody, a labeled detection mAb, astreptavidin-enzyme conjugate HRP and a purified apolipoprotein as a standard.

In other embodiments, apolipoprotein gene expression is measured using, for example, RT-PCR. In certain instances, ApoA1 transcription can be altered by the underlying disorders and the levels of mRNA in an individual's sample can be predictive of the individual's risk of a DILI.

In one embodiment, a method of identifying a patient at risk of a liver injury, and in particular a drug-induced liver injury, is provided comprising 1) measuring the level of apolipoprotein in a bodily fluid from the patient; and 2) comparing the measured level of apolipoprotein in the sample with an ULN in the patient population. A value greater than the ULN is a predetermined level of apolipoprotein that is used as a reference level for determining risk of a liver injury after a patient is administered a drug. In one embodiment the apolipoprotein is ApoA1. Other apolipoproteins are contemplated in the methods described herein.

In one embodiment, the drug is a monoester of probucol, for example the monosuccinic acid ester of probucol.

In another embodiment, a method of identifying a patient at risk of a liver injury, and in particular a drug-induced liver injury, is provided comprising 1) measuring the level of ApoA1 in a bodily fluid from the patient; and 2) comparing the measured level of ApoA1 in the sample to the reference level of ApoA1. If the measured level of ApoA1 is higher that the reference level, the patient may be at greater risk for liver injury than a patient with an ApoA1 level less than or equal to the reference level. It is then possible to exclude a patient from drug treatment using this information.

Methods are also provided for assessing or screening for liver injury, damage or disease in a human is provided by 1) measuring the level of apolipoprotein, such as ApoA1 in a bodily fluid from the patient; and 2) comparing the measured level of apolipoprotein in the sample with an ULN in the patient population, where an apolipoprotein level higher than the ULN indicates liver injury, damage or disease.

In another embodiment, a method is provided for assessing or screening for liver injury, damage or disease in a human is provided by 1) measuring the level of apolipoprotein such as ApoA1 in a bodily fluid from the patient; and 2) comparing the measured level of apolipoprotein in the sample to a predetermined reference level of apolipoprotein where an apolipoprotein level higher than the ULN indicates liver injury, damage or disease.

In one embodiment, a method is provided for diagnosing hepatic events in a human, is provided by 1) measuring the level of apolipoprotein in a bodily fluid from the patient; and 2) comparing the measured level of apolipoprotein in the sample with an ULN in the patient population where an apolipoprotein higher than the ULN indicates a hepatic event.

In another embodiment, a method is provided for assessing or screening for liver injury, damage or disease in a human is provided by 1) measuring the level of apolipoprotein in a bodily fluid from the patient; and 2) comparing the measured level of ApoA1 in the sample to a predetermined reference level of apolipoprotein where an apolipoprotein level higher than the ULN indicates liver injury, damage or disease.

In certain instances, a patient who has one or more samples in which the measured level of apolipoprotein such as ApoA1 is greater than ULN is considered at increased risk of liver injury, in particular at greater risk of drug-induced liver injury.

A patient who has one or more samples in which measured apolipoprotein level, such as ApoA1, exceeds ULN may also be considered to have increased inflammatory activity. Such a patient may be considered at risk for additional disorders, including inflammatory disorders such as rheumatoid arthritis. In certain instances, the patient is at risk of or suffering from a disorder in glucose metabolism. Such a disorder may be diabetes mellitus and in particular may be type 2 diabetes mellitus.

In certain instances, a treatment protocol will be designed based on the results of an ApoA1 measurement. This treatment protocol may require that antioxidant drugs not be given to a patient whose ApoA1 measurement exceeds ULN, or it may be that the patient is closely monitored for hepatotoxicity. In addition, the treatment protocol may require an adjustment in external factors, such as diet or exercise, to decrease additional exposure to environmental toxins that may exacerbate liver injury.

In another embodiment, a method of identifying patients for drug treatment is provided comprising measuring ApoA1 levels in a bodily fluid; comparing the measured value with an ULN of ApoA1 levels in a population; and only providing drug treatment if ApoA1 levels in the fluid are less than or equal to ULN.

In specific embodiments, the patient sample is a serum sample. In other embodiments, the sample is a plasma sample.

In some embodiments, the methods further comprise measuring a level of ALT in a sample from the patient and comparing that to a reference ALT level in the population. In these instances, a measurement of ALT that exceeds ULN is also used to classify the patients as at increased risk of an adverse liver injury. In certain instances, the measurement of ALT is taken before any drug is administered. In these instances, the ALT level can be used as a further exclusionary criteria to identify patients who are at increased risk of a drug induced liver injury. In some instances, an ALT level of greater than ULN is identified, but in other instances an ALT level of at least 1.5 or at least 2.0 or greater ULN is provided as criteria for exclusion. In certain instances, ALT levels are measured in a patient receiving a drug after a period of time, such as at one week, two weeks, three weeks, four weeks, five weeks or more after commencing a therapeutic regimen. Patients whose ALT level is measured as exceeding ULN may be considered at increased risk of, or suffering from, liver toxicity.

Drug-induced liver injury can occur in patients who have been treated with one or more drugs. A wide variety of drugs can induce liver injuries or damage, including but not limited to PPAR agonists, anti-inflammatory drugs, HIV protease inhibitors, neurological drugs, estrogenic and anti-estrogenic drugs, anti-angina drugs, muscle relaxants, anti-psychotic drugs, antihistamines, and other drugs, compounds, and therapeutic agents. In certain embodiments, the drug is an anti-cancer, anti-bacterial, anti-fungal, anti-viral, anti-hypertension, anti-depression, anti-anxiety, and anti-arthritis agent. In another embodiment, the drug is for the treatment of allergies, diabetes, hypercholesteremia, osteoporosis, Alzheimer's disease, Parkinson's disease, and/or other neurodegenerative diseases, and obesity.

Non-limiting examples of PPAR agonists include Pioglitazone, Rosiglitazone, Tesaglitazar, Ragaglitazar, Troglitazone, Farglitazar, Ciglitazone, Azelaoyl PAF, 2-Bromohexadecanoic acid, Clofibrate, 15-Deoxy-d12,14-prostaglandin, Fenofibrate, Fmoc-Leu-OH, GW1929, GW7647, 8 (S)-Hydroxy-(5Z, 9E, 11Z, 14Z)-eicosatetraenoic acid (8(S)-HETE), Leukotriene B4, LY-171,883 (Tomelukast), Prostaglandin A2, Prostaglandin J2, Tetradecylthioacetic acid (TTA), WY-14643 (Pirinixic acid), and NN622 (Novo Nordisk, A/S), and related substances.

Non-limiting examples of anti-anxiety and anti-psychotic drugs include Hydroxyzine Hydrochloride, Lorazepam, Buspirone Hydrochloride, Pazepam, Chlordiazepoxide, Meprobamate, Oxazepam, Trifluoperazine, Clorazepate Dipotassium, Diazepam, Clozapine, Prochlorperazine, Haloperidol, Thioridazine, Thiothixene, Risperidone, Trifluoperazine Hydrochloride, Chlorpromazine, and related substances. Non-limiting examples of HIV protease inhibitors include Saquinavir, Amprenavir, Ritonavir, Nelfinavir, Indinavir, Atazanavir (BMS232632; Bristol-Myers Squibb), Fosamprenavir (GW433908; GlaxoSmithKline), L-756,423 (Merck), Mozenavir (DMP450; Triangle Pharmaceuticals), Tipranavir (PNU-140690; Boehringer Ingelheim); R0033-4649 (Roche) TMC114 (Tibotec Virco), and related substances.

Non-limiting examples of anti-inflammatory drugs include Diclofenac, Diflunisal, Etodolac, Fenoprofen, Flurbiprofen, Ibuprofen, Indomethacin, Ketoprofen, Ketorolac, Meclofenamate, Mefenamic Acid, Nabumetone, Naproxen, Oxaprozin, Piroxicam, Sulindac, Tolmetin, and related substances. Non-limiting examples of antihistimines include Azelastine (Astelin), Fexofenadine (e. g., Allegra), Cetirizine (e. g., Zyrtec, Desloratadine (e. g., Clarinex), Loratadine (e. g., Claritin, Alavert), Astemizole, Azatadine, Brompheniramine, Chlorpheniramine, Clemastine, Cyproheptadine, Dexchlorpheniramine, Dimenhydrinate, Diphenhydramine, Doxylamine, Hydroxyzine, Phenindamine, Pyrilamine, Terfenadine, Tripelennamine, Triprolidine, Methdilazine, Promethazine, Trimeprazine, Diphenhydramine Liquid, and related substances. Non-limiting examples of muscle relaxants include Dantrolene (e. g., Dantrium), Baclofen (e. g., Lioresal, Carisoprodol (e. g., Soma;), Chlorphenesin (e. g., Maolate;), Chlorzoxazone (e. g., Paraflex), Cisatracurium, Cyclobenzaprine (e. g., Flexerilt)), Dantrolene, Diazepam (e. g., Valium;), Metaxalone (e. g., Skelaxin;), Gallamine, Methocarbamol (e. g., Robaxin;), Mivacurium, Orphenadrine (e. g., Norflex), Pancuronium, Rocuronium, Tizanidine, Suxamethonium, Vecuronium, and related drugs.

Non-limiting examples of estrogens and anti-estrogens include conjugated estrogens (e. g., Premarin, esterified estrogens (e. g., Estratabg, Menestg, Estratest;), synthetic conjugated estrogens (e. g., Cenestin), Estropipate (e. g., Ogen, Ortho-Est), Ethinyl Estradiol (e. g., Estinyl), Desogestrel, Diethylstilbestrol (e. g., Stilphostrol), Dienestrol (e. g. , Ortho Dienestrol), Chlorotrianisene (Tace, Estradiol (e. g., Estrace, Alora, Climara, Vivelle), Estradiol Cypionate (e. g., Depo-Estradiolg, Depogens, Dura-Estring, Estra-De, Estro-Cyp, Estroject-LA, Estronol-LA), Estropipate, Ethacrynic Acid, Ethynodiol Diacetate, Levonorgestrel, Medroxyprogesterone, Medroxyprogesterone Acetate, Mestranol, Norethindrone, Norgestimate, Norgestrel, Tamoxifen (e. g., Nolvadex), Toremifene (e. g., Fareston;), Raloxifene (e. g., Evista), Megestrol Acetate (Megace, Aminogluthethimide (e. g., Cytadren), Anastrozole (e. g., Arimidex;), Letrozole (e. g., Femara, Exemestane (e. g., Aromasin), Goserelin (e. g., Zoladex, Leuprolide (e. g., Lupron), and related substances.

Non-limiting examples of anti-angina drugs include Calan SR, Isoptin, Isoptin SR, Verelan, Nicardipine Hydrochloride, Diltiazem Hydrochloride, Nadolol, Isosorbide Mononitrate, Isosorbide Dinitrate, Metroprolol Tartrate, Nitroglycerin, Amlodipine Besylate, Nifedipine, Atenolol, and related drugs.

In some embodiments, the drug induced liver injury is from an antioxidant drug. In certain other instances, the drug induced liver injury is from a drug that increases PPAR activity.

These methods may be used to identify a patient at risk for an adverse hepatic event or presently having an adverse hepatic event when administered a drug to treat a disease. The disease is not critical for the methods described herein and diseases for which drugs are administered may be grouped into three main categories: neoplastic disease, inflammatory disease, and degenerative disease.

Examples of diseases include, but are not limited to, metabolic diseases (e.g., obesity, cachexia, diabetes, anorexia, etc.), cardiovascular diseases (e.g., atherosclerosis, ischemia/reperfusion, hypertension, myocardial infarction, restenosis, cardiomyopathies, arterial inflammation, angina, etc.), immunological disorders (e.g., chronic inflammatory diseases and disorders, such as Crohn's disease, inflammatory bowel disease, reactive arthritis, rheumatoid arthritis, osteoarthritis, including Lyme disease, insulin-dependent diabetes, organ-specific autoimmunity, including multiple sclerosis, Hashimoto's thyroiditis and Grave's disease, contact dermatitis, psoriasis, graft rejection, graft versus host disease, sarcoidosis, atopic conditions, such as asthma and allergy, including allergic rhinitis, gastrointestinal allergies, including food allergies, eosinophilia, conjunctivitis, glomerular nephritis, certain pathogen susceptibilities such as helminthic (e.g., leishmaniasis) and certain viral infections, including HIV, and bacterial infections, including tuberculosis and lepromatous leprosy, etc.), myopathies (e.g. polymyositis, muscular dystrophy, central core disease, centronuclear (myotubular) myopathy, myotonia congenita, nemaline myopathy, paramyotonia congenita, periodic paralysis, mitochondrial myopathies, etc.), nervous system disorders (e.g., neuropathies, Alzheimer's disease, Parkinson's disease, Huntington's disease, amyotropic lateral sclerosis, motor neuron disease, traumatic nerve injury, multiple sclerosis, acute disseminated encephalomyelitis, acute necrotizing hemorrhagic leukoencephalitis, dysmyelination disease, mitochondrial disease, migrainous disorder, bacterial infection, fungal infection, stroke, aging, dementia, peripheral nervous system diseases and mental disorders such as depression and schizophrenia, etc.), oncological disorders (e.g., leukemia, brain cancer, prostate cancer, liver cancer, ovarian cancer, stomach cancer, colorectal cancer, throat cancer, breast cancer, skin cancer, melanoma, lung cancer, sarcoma, cervical cancer, testicular cancer, bladder cancer, endocrine cancer, endometrial cancer, esophageal cancer, glioma, lymphoma, neuroblastoma, osteosarcoma, pancreatic cancer, pituitary cancer, renal cancer, and the like) and ophthalmic diseases (e.g. retinitis pigmentosum and macular degeneration). The term also includes disorders, which result from oxidative stress, inherited cancer syndromes, and metabolic diseases.

The methods and kits of the present invention may also be useful for monitoring and diagnosing various liver diseases, including early stage tissue injury/organ rejection, certain forms of viral infection, drug toxicity, and alterations in liver function. The methods provide information not currently available in the clinical arena, and are rapid and reproducible. The methods and kits are especially useful to evaluate therapeutic agents and drugs for their toxicity with respect to liver damage. The early detection of liver disease by the methods of the present invention can additionally permit earlier clinical intervention if adverse reactions do occur.

### Kits

The present invention also provides kits for determining or predicting in vivo hepatoxicity in a patient or patient population prior to or during administration of a drug, compound, or other therapeutic agent. Such kits are useful in clinical or pre-clinical settings, and can be used concurrently with various stages of patient trials.

In one embodiment, a kit is provided for identification of a patient at risk of a drug induced liver injury comprising a detection system to measure a level of apolipoprotein such as ApoA1 in a patient sample and a system to compare the measured levels to a normal level in a population. The patient sample may be in the form of a bodily fluid, such as blood and blood plasma, mucus, saliva, serum, or urine. In some embodiments, the detection system in the kit can be a labeled antibody to ApoA1 or an ELISA kit comprising a measuring antibody to ApoA1 and a labeled secondary antibody. In other embodiments, the detection system can be a binding partner other than an antibody to apolipoprotein. In yet further embodiments, the detection system can detect levels of the apolipoprotein gene product, such as by RT-PCR. The comparison system can be a separate detection kit in which the level of ApoA1 is standardized to correspond to an upper limit of normal. The readout can be on a colorimetric scale or can be based on a direct comparison of the level of signal from the detection systems. In other embodiments, a chart is included in the kit that allows comparison of the measured ApoA1 levels in the sample with an upper limit of normal in the population.

In certain instances, a visual readout is included which provides a marker signal if the ApoA1 level in the sample is greater than 1.0 times the upper limit of normal. In specific embodiments, the kit includes a detection apparatus that provides a marker signal if the measured level of ApoA1 in the sample is greater than 165 mg/dl. In one embodiment, the predetermined level is a part of the kit so that a minimum concentration of apolipoprotein is required for the kit to identify a positive result. Only individuals having an apolipoprotein concentration greater than the predetermined level will show a positive result when using the kit.

In one embodiment, the kits contain compositions of strips of a solid phase material coated with one or more of the antibodies and are referred to herein as “dipsticks”. The dipsticks specifically bind an apolipoprotein when dipped into a protein sample. The amount of apolipoprotein bound on the dipstick is quantitated using an appropriate method, for example, by staining with a lipid stain or reaction with a second labeled antibody. The intensity of the stain on the dipstick is proportional to the concentration of the apolipoprotein circulating in the blood and can be quantitated by comparison with standards containing known amounts of lipid. The dipsticks can be provided alone or in kits which enable the lay person to carry out the assay without the need of a physician or technical laboratory. In one embodiment, the concentration of anti-apolipoprotein antibody, or other binding element on the dipstick is only sufficient to detect a concentration of apolipoprotein greater than the predetermined level. In this regard, a positive result on the dipstick will only appear when a concentration of apolipoprotein in the test sample exceeds the predetermined level.

Monoclonal antibodies to apolipoproteins can be used not only as components of dipsticks, but also in a variety of other dignostic kits, including enzyme immunoassays, radioimmunoassays as well as fluorescent and chemiluminescent immunoassays to determine apolipoproteins in biological samples with which they are immunoreactive.

Antibodies can be bound to a solid phase material for use in assays described herein. Various types of adsorptive materials, such as nitrocellulose, Immobilon™, polyvinyldiene difluoride (all from BioRad, Hercules, Calif.) can be used as a solid phase material to bind the anti-lipoprotein antibodies. Other solid phase materials, including resins and well-plates or other materials made of polystyrene, polypropylene or other synthetic polymeric materials can also be used. In the preferred embodiment for assaying apolipoprotein concentrations, pieces or strips of these materials are coated with one or more antibodies, or functional fragments thereof, directed against specific epitopes of apolipoproteins for use in patient samples. The dipsticks may also be attached to one end of a longer strip of a solid support material, such as plastic, which can serve as a handle for dipping a dipstick into a solution or sample, such as a sample of whole blood, blood plasma, or blood serum. The plastic handle can also serve as a tether so that multiple dipsticks can be attached to a common support. Such a multi-strip design may be particularly useful in a set-up for testing multiple apolipoproteins simultaneously.

Although various sizes of dipsticks are possible, in one embodiment, pieces of the solid phase material that are coated with antibody have the general dimensions of 0.5 cm×0.5 cm and can be attached to the longer solid support strips having general dimensions of 0.5 cm×5 cm. Such dimensions permit an accurate determination of apolipoprotein levels in as little as 100 μL of blood.

The dipsticks useful in the claimed methods contain one or more regions containing immobilized antibodies specific for particular epitopes on apolipoproteins or lipoproteins. Examples of antibody-conjugated diagnostic dipticks are described for example, but not limited to, U.S. Pat. Nos. 7,098,036; 6,808,889, and 6,087,185.

A dipstick may contain more than one antibody so that the single dipstick can be used to detect more than one apolipoprotein. For example, two or more separate pieces of a solid phase material, each coated with an antibody directed against a particular apolipoprotein or lipoprotein, can be attached to a longer strip of solid support to produce a dipstick with two or more separate areas, each specific for a particular apolipoprotein. The means to attach the solid phase material to a solid support should not impair the function of the molecules coated on the solid phase material and must be secure enough to withstand soaking in whole blood, serum, plasma, and the other solutions described herein which are used to wash, stain, and preserve the dipsticks. A preferred method of attaching antibody-coated solid phase material to a longer strip of solid support is to use a glue or cement such an acrylate adhesive (for example, SUPER GLUE™, Super Glue Corporation, Hollis, N.Y.; DURO™, Loctite Corporation, Cleveland, Ohio).

Dipsticks can be designed for quantification of one or more apolipoproteins in a sample from a test patient. In one embodiment, dipsticks designed for quantification of a apolipoprotein contain a single antigen-binding area which is dipped into a sample, stained for bound lipid lipoproteins or apolipoprotein, and visually compared with a set of printed colored standards to determine the concentration of the particular lipoprotein or apolipoprotein.

In addition, dipsticks can be designed for detecting a change in the relative level of particular apolipoproteins in a sample. Dipsticks can be designed for detecting a change in the relative level of specific apolipoproteins which contain two antigen-binding areas, each area coated with a different antibody. After processing the dipstick to detect the apolipoprotein antigens bound by each antibody, the relative intensities of the colors in the two areas of the dipstick are compared as an indication of the relative concentrations of the two antigens in the blood.

A determination of relative levels of specific apolipoproteins can also be made by simultaneously using two separate dipsticks. However, a single dipstick with two antigen binding areas is generally easier to use, especially for the lay person, and an assessment of relative color intensities in two areas in close proximity on a single dipstick is relatively easy to make even for the untrained observer.

In another embodiment, dipsticks are made that contain distinct areas or spots of known amounts of molecules whose levels are to be determined by the dipstick. For example, known amounts of lipid, lipoproteins and/or apolipoproteins are placed on the dipsticks using methods such as those used for attaching antibodies to the solid phase material described above. Such known amounts of lipids, lipoproteins, and apolipoproteins present on dipsticks act as “internal standards”, whose staining intensity can be compared to that in the antigen-binding areas of the dipstick in order to estimate the amount of antigen bound by the antibodies on the dipstick.

Important reagents in these methods include antibodies or functional fragments of the antibodies, which specifically recognize and bind a particular lipoprotein, leaving other lipoproteins in the sample unadsorbed. In order to assay a sample of whole blood, serum or plasma for apolipoproteins, dipsticks are incubated with EDTA-treated or heparinized blood for 2 to 5 minutes at room temperature. After incubation, each strip is washed to remove unbound blood, (for example, under tap water for 0.5 to 1 minute at temperatures not exceeding 40 C. The dipsticks are then stained, for example, by immersing the dipsticks in a solution of stain such as Sudan Red 7B for 2 to 5 minutes at room temperature to stain the lipid present in the bound lipoprotein particles. Excess stain is then removed by an additional wash. Residual moisture or stain may be drawn off by touching an absorbent towel with the edge of dipstick. The “face” of the dipstick, that is, the side of the dipstick containing immobilized antibody, should not be blotted, which might disturb the immobilized antibody and/or bound antigen. After drying, the intensity of the staining can be compared with standardized colored strips to determine the concentration of lipoprotein in the blood.

A number of other lipid stains such as Oil Red O or Sudan Black B can be also used for staining of dipsticks. However, in the preferred embodiment, Sudan Red 7B, also known as Fat Red 7B (Sigma, St. Louis, Mo.), dissolved in a mixture of methanol and NaOH is used because of its high color intensity. In another embodiment lipoproteins are stained prior to being bound to antibody (“pre-stained”), such as antibody on a dipstick, using any of the above mentioned lipid stains dissolved in propylene glycol (Wollenweber, J. and Kahlke, W., Clin. Chim. Acta, 29:411-420 (1970)). The pre-stained blood, plasma or serum sample is then incubated, for example, with anti-LDL or anti-HDL dipsticks. After washing and drying, the quantity of pre-stained lipoprotein captured by the dipstick is determined visually according to the intensity of the color, for example, by comparison with a set of printed colored standards.

Many detectable labels, reporters, moieties are known in the art and can be used with the invention. For example, the detectable moiety may be chromogenic, fluorogenic, or luminescent, or may be a member of a specific binding pair, a substance detectable by an antibody in any of the known immunoassay methods. There are many different labels and methods of labeling known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds. Those of ordinary skill in the art will know of other suitable labels for binding to apolipoprotein such as ApoA1, a compound that binds apolipoprotein or an antibody that binds apolipoprotein, or will be able to ascertain such, using routine experimentation.

Modifications and variations of the present invention will be obvious to those skilled in the art from the foregoing. All of these embodiments are considered to fall within the scope of this invention.

### EXAMPLES

Analyses were conducted to identify potential risk factors for drug-induced liver toxicity in clinical studies of the monosuccinic acid ester of probucol (AGI-1067). AGI-1067 represents a new class of therapies targeting certain chronic diseases such as type II diabetes mellitus and atherosclerosis. AGI-1067 functions both as a direct antioxidant and an inducer of endogenous, antioxidant processes such as hemeoxygenase-1 and thioredoxin.

In a large clinical study (more than 6000 pts, 2 year average exposure), AGI-1067 was shown to reduce hard cardiovascular endpoints (a composite of cardiovascular death, non-fatal myocardial infarction (MI) and non-fatal stroke) in well-treated patients with pre-existing coronary artery disease and to lower HbA1c levels in a large subset of these patients with type 2 diabetes mellitus. In the same study, a small number of the 3078 patients randomized to treatment with AGI-1067 exhibited reversible elevations in liver function tests with eight of those patients demonstrating elevated ALT and bilirubin compared with four patients in the placebo arm.

### Example 1

### ALT Elevation Alone Predicts Depatotoxicity Poorly

There were 36 patients who had peak elevations of ATL between 3× and ≦5× ULN in the combined dataset of diabetes mellitus patients. A summary of the data by treatment group is shown in Table 1. The incidence of ALT elevations in this range was comparable for the AGI-1067 and placebo treated groups of patients. ALT levels therefore appear to have limited value in assessing potential for hepatotoxicity.

TABLE 1

| | | Number of | |
|---|---|---|---|
| | | Patients | |
| | | ALT | |
| | Percent of | 3X to ≦ 5X ULN | Percent |
| | Randomized | With | of ALT |
| | Diabetes Patients | TBL ≦ 2X ULN | Elevations |
| Placebo | 43% | 19 | 53% |
| AGI-1067 | 57% | 17 | 47% |
| 75 mg | 9% | 3 | 8% |
| 150 mg | 9% | 4 | 11% |
| 300 mg | 39% | 10 | 28% |
| Total | | 36 | |

### Example 2

### ALT Elevations in Combination with Total Bilirubin Levels is a Useful Indicator of Hepatic Events

There were 24 diabetes patients in the combined dataset who had hepatic events as using a criteria of either ALT >5× ULN plus TBL <2× ULN or ALT >3× ULN plus TBL >2× ULN criteria. Table 2 summarizes the patients by treatment arm and by dose of AGI-1067. At randomization, 57% of the patients were in the AGI-1067 arm and 43% were in the placebo arm resulting in an AGI-1067 to placebo ratio of 1.3. Of the 24 hepatic events, 17 occurred in AGI-1067 treated patients and 7 occurred in placebo treated patients.

TABLE 2
BEFORE USE OF THE RISK IDENTIFICATION TOOL

| | | Number of | | | |
|---|---|---|---|---|---|
| | | Hepatic Events | | | |
| | Percent of | ALT > 5X | ALT > 3X | | |
| | Randomized | ULN With | ULN Plus | | Percent of |
| | Diabetes | TBL ≦ 2X | TBL > 2X | | Hepatic |
| | Patients | ULN | ULN | Total | Events |
| Placebo | 43% | 4 | 3 | 7 | 29% |
| AGI-1067 | 57% | 13 | 4 | 17 | 71% |
| 75 mg | 9% | 4 | 0 | 4 | 17% |
| 150 mg | 9% | 2 | 0 | 2 | 8% |
| 300 mg | 39% | 7 | 4 | 11 | 46% |
| Total | | 17 | 7 | 24 | |

### Example 3

### ApoA1 Levels are Directly Correlated to Adverse Liver Events

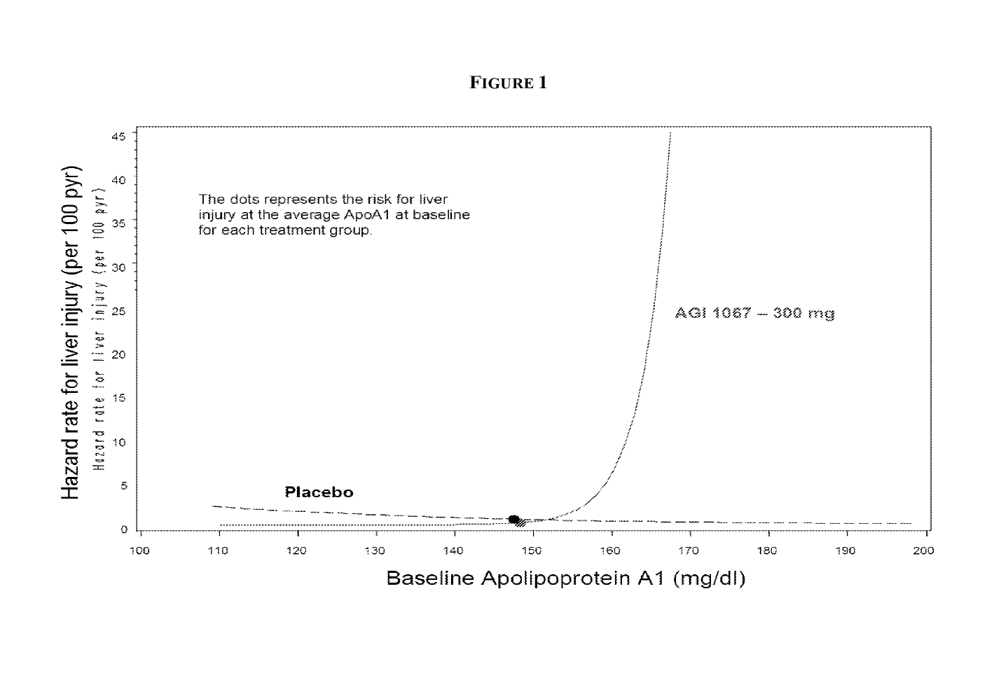

ApoA1 measurement was both sensitive and specific for identifying subsequent hepatic events. The events were defined as a measurement of ALT >5× ULN with TBL <2× ULN or ALT >3× ULN with TBL >2× ULN. FIG. 1 was generated from type 2 diabetes mellitus patient data shows age-adjusted effect for the 5th to 95th percentile range of baseline ApoA1 on subsequent liver events using a Cox Proportional Hazards Model. As a point of reference, the ULN for ApoA1 was 165 mg/dL for this trial. The solid line and the dashed line represent AGI-1067 and placebo data, respectively.

### Example 4

### Measurement of ApoA1 Provides a Useful Tool for Identifying Patients at Risk of Drug Induced Liver Toxicities

As noted in Example 2, hepatic events in the sample populations were distributed 71% (17/24) for AGI-1067 treated patients and 29% (7/24) for patients receiving placebo resulting in an AGI-1067 to placebo ratio of 2.4. When patients in whom ApoA1 measurements before administration of any drug exceeded ULN (here, 165 mg/dl) were excluded from the sample, the number of hepatic events achieved parity for the AGI-1067 and placebo treatment arms.

There were 14 patients in the combined dataset who had hepatic events using the ALT >5× ULN plus TBL <2× ULN or ALT >3× ULN plus TBL >2× ULN criteria. Table 3 summarizes the patients by treatment arm and by dose of AGI-1067. As noted in Example 2, at randomization the ratio of AGI-1067 to placebo patients was 1.3, the same value as the randomization ratio for AGI-1067 compared to placebo.

When patients with elevated ApoA1 levels or ALT levels of greater than 2.0 times ULN were excluded, 10 hepatic events were eliminated. Of these, 90% were in the AGI-1067 treatment arm. Of the remaining 14 hepatic events, 8 were in AGI-1067 treated patients while 6 were in placebo treated patients. These hepatic events were distributed 57% (8/14) for AGI-1067 patients and 43% (6/14) for patients receiving placebo. This resulted in an AGI-1067 to placebo ratio of 1.3.

TABLE 3
USING THE RISK IDENTIFICATION TOOL

| | Percent of | Number of Hepatic Events | | | Percent |
|---|---|---|---|---|---|
| | Randomized | ALT > 5X ULN | ALT > 3X ULN | | of |
| | Diabetes | With | Plus | | Hepatic |
| | Patients | TBL ≦ 2X ULN | TBL > 2X ULN | Total | Events |
| Placebo | 43% | 3 | 3 | 6 (↓14%) | 43% |
| AGI-1067 | 57% | 6 | 2 | 8 (↓53%) | 57% |
| 75 mg | 9% | 2 | 0 | 2 | 14% |
| 150 mg | 9% | 1 | 0 | 1 | 7% |
| 300 mg | 39% | 3 | 2 | 5 | 36% |
| Total | | 9 | 5 | 14 (↓42%) | |

Thus, application of the exclusionary criteria resulted in very low rates of hepatic events with an incidence of 0.4% for both the AGI-1067 (8/1851) and placebo (6/1419) groups.

Table 4 summarizes the impact of the exclusionary criteria (primarily ApoA1 elevation) on the number, frequency distribution and ratio of hepatic events for the AGI-1067 and placebo groups in the 3270 type 2 diabetes mellitus patients contained in the datasets.

TABLE 4

| | | Before Use | | | |
|---|---|---|---|---|---|
| | | of the Risk | | Using the Risk | |
| | Percent of | Identification Tool | | Identification Tool | |
| | Randomized | Number of | Percent of | Number of | Percent of |
| | Diabetes | Hepatic | Hepatic | Hepatic | Hepatic |
| | Patients | Events | Events | Events | Events |
| Placebo | 43% | 7 | 29% | 6 | 43% |
| AGI-1067 | 57% | 17 | 71% | 8 | 57% |
| Ratio | 1.3 | 2.4 | | 1.3 | |
| 1067/ | | | | | |
| Placebo | | | | | |
| Hepatic | | | | | |
| Events | | | | | |

Table 5 shows that using Baseline ApoA1 as a “Predictive Biomarker” would have resulted in seven times more excluded hepatic events for the AGI-1067 patients than for the placebo patients. ALT >2× ULN could also be used as a secondary indicator for exclusion of patients.

TABLE 5

Number of Hepatic
Events Excluded

| Biomarker | AGI-1067 | Placebo |
|---|---|---|
| Apo A1 > 165 mg/dl | 7 | 1 |
| Baseline ALT > 2X ULN | 1 | 0 |
| Month 1 ALT > 2X ULN | 1 | 0 |
| Totals | 9 | 1 |

1. A method of treating a patient with a pharmacological agent that can induce liver toxicity comprising:
a) measuring the level of apolipoprotein in a bodily fluid from the patient; and
b) comparing the measured level of apolipoprotein in the sample with a reference value in a patient population;
c) wherein the patient is administered a pharmacological agent if the measurement is less than or equal to the reference value.

2. The method of claim 1 wherein the pharmacological agent is administered for treatment of diabetes in a patient in need thereof

3. The method of claim 1 wherein the pharmacological agent is administered for treatment of a cardiovascular disease in a patient in need thereof.

4. The method of claim 3 wherein the pharmacological agent is a monoester of probucol.

5. The method of claim 1 wherein the apolipoprotein is selected from Apo A-I, Apo A-II, Apo A-IV, Apo B-100, Apo B-48, Apo C-I, Apo C-II, Apo C-III or Apo E.

6. The method of claim 1 wherein the apolipoprotein is Apo-A1 or structurally modified Apo-A1.

7. The method of claim 6 wherein the reference value for Apo-A1 is between about 155 and 195 mg/dL.

8. The method of claim 7 wherein the reference value for Apo-A1 is 165 mg/dL.

9. The method of claim 1 further comprising measuring at least one of a level of ALT in the patient and a level of total bilirubin in the patient and comparing the measured level to a reference value of ALT or a reference level of total bilirubin in the population.

10. The method of claim 9 wherein the pharmacological agent is only administered to the patient if the measured level of ALT is at least 2 times the reference value or if the measured level of total bilirubin is at least above 1 times the reference value.

11. The method of claim 1 wherein the bodily fluid is selected from blood, blood plasma, mucus, saliva, serum, or urine.

12. A kit for identification of a patient at risk of a drug induced liver injury comprising: a) a detection system to measure a level of apolipoprotein in a patient sample; and b) a system to compare the measured levels to a predetermined upper limit of normal (ULN) in a population.

13. The kit of claim 12 wherein the detection system is fixed on a solid support.

14. The kit of claim 13 wherein the detection system comprises antibodies to apolipoprotein.

15. The kit of claim 12 wherein the kit comprises a visual readout signal if the apolipoprotein level in the sample is greater than 1.0 times the ULN.

16. The kit of claim 12 wherein the apolipoprotein is ApoA1.

17. The kit of claim 12 further comprising a system for measuring a level selected from a level of ALT in the sample, a level of total bilirubin in the sample, and a combination of ALT and total bilirubin in the sample and comparing the measured level to a ULN for ALT or total bilirubin in a population.

18. The kit of claim 17 comprising a visual readout signal if the level of ALT is greater than about 2 times ULN for ALT or if the level of total bilirubin is greater than about 1 times ULN for total bilirubin.

19. The kit of claim 16 wherein the ULN for Apo-A1 is between about 155 and 195 mg/dL.

20. The kit of claim 16 wherein the ULN for ApoA1 is 165 mg/dl.

21. The method of claim 1 wherein the pharmacological agent is administered for treatment of a metabolic disease in a patient in need thereof.

22. The method of claim 1 wherein the pharmacological agent is administered for treatment of a disorder in glucose metabolism in a patient in need thereof.

23. The method of claim 2, wherein diabetes is type 2 diabetes mellitus.

24. The method of claim 4, wherein the monoester of probucol is the monosuccinic acid ester of probucol.

25. The method of claim 6, wherein the structurally modified Apo-A1 is a lipid modified Apo-A1.

26. In a method for treating a patient with type II diabetes with a pharmaceutically effective formulation of the monosuccinic acid ester of probucol, the improvement comprising administering the formulations only to a patient who has been tested for Apo-A1 level and has an Apo-A1 level at or below a reference level of about 165 mg/dl.

27. In a method for treating a patient with type II diabetes with a pharmaceutically effective formulation of the monosuccinic acid ester of probucol, the improvement comprising administering the formulations only to a patient that has an Apo-A1 level at or below a reference level of about 165 mg/dl.
